(19) United States
(12) Patent Application Publication
(10) Pub. No.: US 2010/0016257 A1
(43) Pub. Date: Jan. 21, 2010

(54) METHOD OF TREATMENT FOR OSTEOARTHRITIS BY LOCAL INTRA-ARTICULAR INJECTION OF MICROPARTICLES

(75) Inventors: Laura J. Brown, Hamilton Square, NJ (US); Han Cui, Bridgewater, NJ (US); Zhangwen Wu, New Hope, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: Ethicon, Inc., Somerville, NJ (US)

(21) Appl. No.: 12/564,237

(22) Filed: Sep. 22, 2009

Related U.S. Application Data
(62) Division of application No. 11/300,822, filed on Dec. 15, 2005.

(51) Int. Cl. A61K 31/728; A61P 19/02

(52) U.S. Cl. 514/54

(57) ABSTRACT

A method of treatment of osteoarthritis is described, where a therapeutically effective amount of a composition having biodegradable microparticles in an aqueous vehicle is delivered into the intra-articular space of a joint. In one aspect, the microparticle-containing composition is injected into the synovial fluid-containing portion of an affected joint.

### FIELD OF THE INVENTION

The present invention relates to a method of treatment for osteoarthritis (OA), more specifically, the use of biodegradable microparticles in an aqueous vehicle as an intra-articularly delivered disease-modifying treatment for osteoarthritis.

### BACKGROUND OF THE INVENTION

Osteoarthritis (OA), also known as degenerative joint disease, is the most common form of arthritis and results from the gradual breakdown of cartilage that accompanies aging. Typically, OA follows trauma or chronic joint injury due to some other type of arthritis such as rheumatoid arthritis. Alternatively, OA can result from overuse of a particular joint. OA most commonly involves the joints of the elbow, fingers, hips, knees, shoulder, wrist, spine, and toes. Clinically, OA is characterized by joint pain, tenderness, limitation of movement, crepitus, and inexorably progressive disability. It can be present in just one of these joints or in all of them. Although most body tissues can make repairs following an injury, it is believed cartilage repair is hampered by a limited blood supply and the lack of an effective mechanism for cartilage re-growth.

Historically, conventional treatment of osteoarthritis injuries has been limited to pain relief, reduction of joint loading, physical therapy, and orthopedic surgery, all of which are aimed at symptomatic relief rather than disease-modifying treatment of the underlying pathologic disorder. One currently used conventional treatment regimen for arthritis includes oral delivery of first line drugs for control of pain and inflammation classified as non-steroidal anti-inflammatory drugs (NSAIDs), such as, aspirin, ibuprofen, and naproxen, etc. Secondary treatments include corticosteroids or slow acting anti-rheumatic drugs (SAARDs). Although NSAIDS are one of the major groups of drugs in terms of sales and use for the management of OA among the general population, there may be certain disadvantageous side effects, particularly in the elderly. Virtually all NSAIDS are believed to cause gastrointestinal hemorrhage, ulceration, or perforation, while some may be associated with bone marrow depression, several may cause fluid retention, and may contribute to renal failure. These side effects must be carefully weighed against the advantages because such treatments are often long-term since the indications are often chronic.

While the previously mentioned drugs have met with a certain degree of success in the prevention and treatment of pain associated with osteoarthritis, new and improved methods and pharmaceutical compositions are constantly being sought which may effectively reduce the progression of lesion and cartilage degradation in a mammal suffering from osteoarthritis.

Known pharmaceutical compositions and methods of local treatment of osteoarthritis have contained hyaluronic acid (HA) as the active ingredient.

It is believed that the use of HA as an osteoarthritis therapy provides temporarily relief of chronic symptoms such as joint pain and stiffness as a result of its viscosupplementation properties. However, at the present time it is generally believed that the use of HA as the sole active agent for treating osteoarthritis does not directly relieve chronic symptoms or modify the progression of the disease. In addition, HA has a relatively limited residence time, and the patient will typically require additional doctor visits for repeated treatments.

Therefore, there is continuing need in this art for novel disease-modifying treatments for osteoarthritis that can be delivered to the OA affected site.

### SUMMARY OF THE INVENTION

Accordingly, a novel method of treating osteoarthritis is disclosed.

In the method of treatment of the present invention, a therapeutically effective amount of a sterile composition containing biodegradable microparticles in an aqueous injection or carrier vehicle is injected or delivered into the intra-articular space of an osteoarthritic joint. The aqueous vehicle optionally contains a viscosity enhancer such as hyaluronic acid.

Another aspect of the present invention is a composition for treating osteoarthritis. The composition consists of an aqueous injection or carrier vehicle and biodegradable microparticles. The composition may optionally contain viscosity enhancers. The composition may be injected or infused into the intra-articular space of a joint.

The methods and compositions of the present invention may be used as a disease-modifying treatment of osteoarthritis when injected, infused or otherwise delivered directly into an affected joint.

These and other aspects of the present invention will become more apparent from the following description and examples.

### DETAILED DESCRIPTION OF THE INVENTION

Biodegradable microparticles have been extensively and effectively used as controlled release systems for therapeutic agents including active pharmaceutical ingredients (APIs) and macromolecules. Since they afford sustained release of the encapsulated material, their use in the encapsulation of therapeutic agents could improve the site-specificity over a controlled duration and reduce any toxic systemic effects. So, the concept of drug-loaded microparticles intra-articularly injected into an osteoarthritis site has been studied as a possible means of treating osteoarthritis related symptoms.

It is known, however, that macrophages will become activated in response to particulate biomaterials when the implanted biomaterial is in the size range between 20 to 60 microns. So, it is expected that microparticles intra-articularly injected into an osteoarthritis site would negatively affect the osteoarthritis-related symptoms. Surprisingly, a formulation of the present invention comprised of biodegradable microparticles (without added therapeutic agent) in an aqueous vehicle when intra-articularly injected or infused into an osteoarthritis site is extremely well tolerated. Even more surprisingly, the formulation of the composition used in the method of the present invention provides a disease-modifying treatment for OA. Therefore, the method of the present invention for treatment of OA provides for injecting or otherwise infusing biodegradable microparticles in an aqueous vehicle into the intra-articular space of an affected joint. It is also possible that the method of the present invention may provide a certain degree of prophylaxis.

Affected joints may be any joint in the body and include, but are not limited to, the hip, knees, shoulders, ankles, elbows, wrists, toes, fingers, and spinal facet joints. Each of these joints have opposing bones having respective opposing hyaline cartilage articular surfaces; a peripheral, collagenous ligamentous capsule connecting the articular surfaces and defining a central joint space; a synovium lining upon an inner wall of the capsule, and synovial fluid contained within the joint space.

The method of the present invention for treatment of OA provides for injecting or otherwise infusing biodegradable microparticles in an aqueous vehicle such as an injection vehicle into the intra-articular space of an affected joint in order to access an OA-affected area or treatment site. Preferably, the direct administration includes depositing the biodegradable microparticles in an aqueous injection vehicle into the synovial fluid containing portion of the joint through a small gauge needle.

The therapeutically effective amount of formulation injected into an affected area or treatment site is dependent on several factors, including but not limited to location of the OA site and the size of the affected joint. For example, a therapeutic amount of preferably about 2 milliliters of the formulation will be injected or infused into the human intra-articular space of the knee. Suitable volume will be easily adjusted by one of ordinary skill in this art for injections or delivery into other joints, such as the hip, shoulders, ankles, elbows, wrists, toes, fingers, and spinal facet joints.

It will be appreciated by those skilled in this art that although injection by syringe is the preferred delivery method for the compositions of the present invention, other conventional modalities for delivering the compositions to a treatment site may be used as well. The other conventional delivery modalities include catheters, infusion pumps, pen devices and the like.

The biocompatible, biodegradable microparticles that can be used in the practice of the present invention can be made from conventional natural and synthetic materials and may be polymers. A biocompatible material is defined as a material that is not toxic to the human body, it is not carcinogenic and it should induce limited or no inflammation in body tissues. A biodegradable material is defined as a material that is degraded by bodily processes (e.g., enzymatic) to products readily disposable by the body or absorbed into body tissue. The biodegraded products should also be biocompatible with the body. Preferably, the biodegradable microparticles are polymers.

Suitable examples of biocompatible, biodegradable polymers that may be used to make the biodegradable microparticles of the present invention include but are not limited to poly(alpha-hydroxy acid) polymers such as poly(lactic acid) (PLA), poly(glycolic acid) (PGA, copolymers of lactic acid and glycolic acid (PLGA), polyoxalates, polycaprolactone (PCL), copolymers of caprolactone and lactic acid (PCLA), poly(ether ester) multiblock copolymers based on poly(ethylene glycol) and poly(butylene terephthalate), tyrosine-derived polycarbonates, poly(hydroxybutyrate), polydioxanone, poly(alkylcarbonate), poly(orthoesters), polyesters, poly(hydroxyvaleric acid), poly(malic acid), poly(tartaric acid), poly(acrylamides), polyanhydrides, and polyphosphazenes. Suitable polymeric materials also include waxes such as glycerol mono- and distearate and the blends thereof.

The biodegradable polymers listed above typically have a hydrophilic end group such as carboxylic acid. Polymers with hydrophobic end groups may be easier to suspend in an aqueous injection vehicle and aid in prevention of agglomeration. Therefore, the biodegradable polymers may be endcapped with a hydrophobic group including, but not limited to, lauryl esters or methoxy.

Preferred biodegradable polymers used in the microparticles of the present invention include poly (alpha-hydroxy acid) polymers such as poly(lactic acid) (PLA), poly(glycolic acid (PGA), and copolymers of lactic acid and glycolic acid (PLGA). More preferably, the polymers are endcapped with hydrophobic groups such as lauryl ester.

The biodegradable microparticles of the present invention can be prepared by any known method and equivalents thereof, that is capable of producing microparticles in a size range sufficiently effective for use in an injectable formulation or for delivery or infusion through a hypodermic needle or catheter. Several conventional methods have been commonly utilized for making biodegradable polymeric microparticles or microspheres. Such methods for making microparticles include double emulsion/solvent evaporation and spray drying.

In the emulsion/solvent evaporation process, a suitable biodegradable polymer is dissolved in an organic solvent resulting in the organic phase.

Suitable organic solvents for the polymeric materials include but are not limited to acetone, halogenated hydrocarbons such as chloroform and methylene chloride, aromatic hydrocarbons such as toluene, halogenated aromatic hydrocarbons such as methylene chloride, and cyclic ethers such as dioxane. The organic phase is then mixed with a non-solvent for the polymer such as an aqueous or silicone based solvent to form an emulsion. The emulsion is then mixed with a larger volume of the non-solvent. The microparticles are then collected and dried. Process parameters such as solvent and non-solvent selections, polymer/solvent ratio, temperatures, stirring speed, and dry cycles are adjustable to achieve the desired particle size, surface smoothness, and narrow particle size distribution. Surfactants such as polyvinylalcohol can be incorporated into the non-solvent to form microparticles with a smoother surface.

In the coacervation process, a suitable biodegradable polymer is dissolved in an organic solvent resulting in the organic phase. Suitable organic solvents for the polymeric materials include but are not limited to acetone, halogenated hydrocarbons such as chloroform and methylene chloride, aromatic hydrocarbons such as toluene, halogenated aromatic hydrocarbons such as methylene chloride, and cyclic ethers such as dioxane. The organic phase is then mixed with a non-solvent for the polymer such as silicone-based solvent. The non-solvent is miscible with the organic solvent and in which the polymer has a low solubility. By mixing, the non-solvent causes the polymer to come out of solution in the form of a dispersed liquid phase comprising polymer droplets. The polymer droplets are then mixed with a hardening agent to form solid microparticles. The microparticles are then collected and dried. Process parameters such as solvent and non-solvent selections, polymer/solvent ratio, temperatures, stirring speed, and dry cycles are adjustable to achieve the desired particle size, surface smoothness, and narrow particle size distribution.

In the spray drying process, a suitable biodegradable polymer is dissolved in an organic solvent, such as listed above, and then sprayed through nozzles into a drying environment provided with sufficient elevated temperature and/or flowing air to effectively extract the solvent. Adding surfactants, such as sodium lauryl sulfate (SLS), TWEENs (polyoxyethylene sorbitan monoleate (Sigma, St. Louis, Mo.)) and PLURONICs (triblock poly(ethylene oxide) (PEO)-poly(propylene oxide) (PPO)-poly(ethylene oxide) (PEO) copolymer (BASF, Worcester Mass.)) can improve the surface smoothness of the microparticles.

A particularly preferred method of microparticle preparation is the coacervation method. A preferred organic solvent is methylene chloride. A preferred non-solvent is a silicone based solvent. Also, after the microparticles are collected, drying is preferably performed under vacuum.

The biodegradable microparticles that can be used in this invention can be of various shapes including but not limited to spherical, pyramidal, cubical, cylindrical, rhombic, and other geometric shapes but are preferably substantially spherical. The particle size range for such particles is preferably sufficient such that they are effectively injectable through a 16 to 24 gauge needle. The preferred mean particle size range for the microparticles is about 5 to 150 microns, and more preferably the mean particle size range is about 10 to 100 microns. The most preferred mean particle size range is 35 to 45 microns. A combination of mean particle size ranges may provide a more long lasting effect such as, a formulation that contains microparticles of a mean particle size range of 30 to 50 microns mixed with microparticles of a mean particle size range of 125 to 150 microns. It will be appreciated by those skilled in the art that the aqueous compositions of the present invention containing microparticles may be delivered to a treatment site by other conventional methods, including catheters, infusion pumps, pens devices and the like. The particle sizes of the microparticles would be adjusted correspondingly.

Carrier vehicles for use in the present invention includes, but is not limited to, water and aqueous solutions of viscosity enhancers. Suitable viscosity enhancers include, but are not limited to, hyaluronic acid, modified hyaluronic acid, sodium hyaluronate solutions for treatment of articular disorders such as those sold under the tradename ORTHOVISC (DePuy Ortho Biotech products, L.P., Raritan, N.J.), collagen, poly(alkylene oxide)-based polymers such as polyethylene glycol, chitosan, fucans, copolymers of polysaccharides with degradable polymers, gelatin, starch, cellulose, cellulose derivatives (e.g., regenerated cellulose, methylcellulose, hydroxypropylcellulose, hydroxypropylmethylcellulose, carboxymethylcellulose, cellulose acetate phthalate, cellulose acetate succinate, hydroxypropylmethylcellulose phthalate), casein, dextrans, polysaccharides, and any viscosupplement formulations. The preferred viscosity enhancers include sodium hyaluronate solutions and modified hyaluronic acid.

The aqueous solutions of the viscosity enhancers are preferably in a concentration of about 0.5 to 5 percent weight/weight (w/w) and more preferably in the range of about 1 to 3 percent (w/w). The most preferred concentration of injection vehicle in aqueous solution is about 1.5 to 2.5 percent (w/w). The viscosity should be sufficient to effectively provide for suspension of microparticles in the carrier vehicle and ease of administration by the delivery methods previously described herein.

The biodegradable microparticle loading in the carrier vehicle can be adjusted such that the formulation is injectable through a 16 to 24 gauge needle, and adjusted similarly if other delivery methods are used. The biodegradable microparticle loading in the injection vehicle can range from 1 to 20 percent weight/weight (w/w), preferably 1.5 to 9 percent (w/w). The size of the needle will be related to the size of the joint that is being treated and the formulation properties. In the case of human knee joints, for example, the preferable needle size is 18 gauge.

The compositions of the present invention having a formulation of biodegradable microparticles in an aqueous injection vehicle for use as a treatment for osteoarthritis can be prepared by any known method. For example, the microparticles prepared as described above are sterilized by common sterilization methods such as ethylene oxide (EtO) exposure. The microparticles are incorporated into a sterile aqueous injection vehicle aseptically. Alternatively, the microparticles could be mixed with the vehicle manually or by other mechanical mixing methods, subsequently loaded into syringes, sterilized, and packaged ready for use.

Preferably, the biodegradable microparticle formulation in an aqueous vehicle should be prepared just prior to use to minimize any premature swelling and degradation of the biodegradable microparticles prior to use. Alternatively, the formulation could be refrigerated after preparation if it will not be used immediately.

The following examples are illustrative of the principles and practice of this invention, although not limited thereto.

### Example 1

### Preparation of the Microparticles

Polymer microparticles (Mean Size=35.1 micron; PLGA 75/25 percent mole/mole (mol/mol) (Alkermes, Cincinnati, Ohio,); Inherent Viscosity (I.V.)=0.61 deciliters/gram in chloroform at 25 degrees Celsius were made using the emulsion/solvent evaporation procedure. 5 grams of polymer were added to 125 grams of methylene chloride (Fisher Scientific, Pittsburgh, Pa.) and mixed for 30 minutes. The polymer solution was added to 185 grams of Dow Corning Medical Fluid with a viscosity of 350 centistokes (Dow Corning, Midland, Mich.) and agitated for approximately 3 minutes to form an emulsion. The emulsion was agitated for an additional 3 minutes then transferred into 2500 grams of cyclomethicone (Rhodia, Cranbury, N.J.) and mixed for approximately 1 hour. Microspheres were then collected on a stainless steel screen and dried under vacuum with gradually elevated temperature.

### Example 2

### Preparation of Osteoarthritis Treatment Formulation

Sterile microparticles from Example 1 were dispersed in hyaluronic acid aqueous carrier vehicle (tradename ORTHOVISC, DePuy Ortho Biotech products, L.P., Raritan, N.J.). The microparticles were aseptically mixed in ORTHOVISC manually with a spatula. The microparticles were added to obtain a particle loading of 1.67 percent (w/w) of microparticles in ORTHOVISC.

### Example 3

### In Vivo Osteoarthritis Treatment Experiment

A rabbit study was performed to study the effectiveness of the injectable formulation of microparticles as a treatment to relieve osteoarthritis related symptoms. In summary:

Female New Zealand White Rabbits (Millbrook Breeding Labs, SPF, Amherst, Mass.) underwent Anterior Cruciate Ligament Transection (ACLT) on the right knee. After six weeks, intra-articular injections were given to the operated knee once per week for five weeks. After the last injection, one more week elapsed prior to euthanasia (a total of 12 weeks from time of ACLT). Animals were euthanized and gross observations were made on the knee joints. The stifle joints were removed. Tissue samples were preserved in 10 percent neutral buffered formalin. Histological sectioning and staining were performed on the condyles.

The study was conducted in accordance with the rules and regulations of the Institutional Animal Care and Use Committee of SUNY Health Science Center at Brooklyn. The rabbits were group housed in pens. Diet consisted of a commercially available rabbit chow and tap water. The rabbits utilized in this study were handled and maintained in accordance with the current requirements of the Department of Animal Laboratory Resources and Maimonides at SUNY.

Animals were weighed and anesthesia was induced in each rabbit via an intra-muscular injection of Ketamine (17 milligram/kilogram) and Xylazine (2.5 milligram/kilogram). Supplementation, during surgery, was given if needed with additional intra-muscular injections of Ketamine (35 milligram/kilogram) or Xylazine (5 milligram/kilogram).

Analgesia in these animals was accomplished with Buprenorphine (0.01-0.05 milligram/kilogram) via a sub-cutaneous injection. The Buprenorphine was administered every 12 hours for 72 hours.

After induction of anesthesia, the right leg skin surface was clipped free of hair using electric animal clippers. The area around the site of surgery was scrubbed with Chlorhexidine diacetate, rinsed with alcohol, dried, and painted with an aqueous iodophor solution of 1 percent available iodine. The anesthetized and surgically prepared animal was placed in the desired recumbent position. Sterile drapes were applied to the prepared area using aseptic technique.

In the right limb of each animal, the ACL was transected. A medial parapatellar incision was made and the patella dislocated. The knee was flexed and the ACL visualized. A scalpel blade was positioned behind the ACL and brought anteriorly, thereby cutting the ACL while protecting the posterior cruciate ligament. The patella was returned to the normal anatomic position. The wound was closed in layers.

Animals were allowed to move freely as soon as they recovered from anesthesia.

Following surgery, all animals were untreated for six weeks. Data generated from previous rabbit studies has shown that six weeks allows for sufficient joint degeneration to occur. This approach mirrors clinical patient presentation for therapeutic intervention. After six weeks, a series of five injections of 160 microliters of injection vehicle control (ORTHOVISC) and microparticle formulation from Example 2 were injected intra-articularly once weekly using a 23 gauge needle. Treatment groups were compared to both the vehicle control and the untreated control groups. In addition, the unoperated, contralateral control stifle joint of each animal was examined for degenerative changes that would be attributed to changes in gait.

The animals were euthanized 6 weeks after initiation of injections with an intravenous injection of pentobarbital (60 milligrams/kilogram). Following administration of the drug, the animals were observed to ensure that respiratory function had ceased and there was no palpable cardiac function.

At the time of sacrifice, femoral condyle effects were evaluated grossly. Percent surface area erosion of cartilage for medial and lateral condyles were scored from 0-5 each (maximum of 10 points toward total score) with scoring as follows: 0=no erosion; 1=less than or equal to 10 percent erosion; 2=11-25 percent erosion; 3=26-50 percent erosion; 4=51-75 percent; 5=76-100 percent erosion. Depth of erosion of cartilage for medial and lateral condyles were scored from 0-3 each (maximum of 6 points toward total score) with scoring as follows: 0=none; 0.5=barely perceptible; 1=slight; 2=significant; 3=severe. Presence of clefts was also evaluated and scored from 0-2 (maximum of 2 points toward total score) with scoring as follows: 0=absent; 1=unicondylar; and 2=bicondylar. The femoral condyle total score is the sum of the five femoral condyle scores described above. Therefore, the range for femoral condyle total score is from 0-18. A high score indicates a negative effect and more tissue damage while a lower score indicates a positive effect or similar to the normal tissue. The femoral condyle effects total score is the most relevant to clinical human osteoarthritis condition.

Table 1, shown below, summarizes the results of the rabbit ACLT study at six weeks after starting the injection regimen. The table lists the treatment groups, number of treatments per group, the mean femoral condyle total scores, and the standard error of the means. The treatment groups include two controls, untreated and treatment with injection vehicle (ORTHOVISC) alone, and microparticle formulation in the injection vehicle.

TABLE 1
Summary of Femoral Condyle Total Scores

| Treatment | | | Standard Error of |
|---|---|---|---|
| Group | N per Group | Mean | the Means |
| Untreated Control | 10 | 5.15 | 4.84 |
| ORTHOVISC | 10 | 4.10 | 3.86 |
| control | | | |
| Microparticle | 10 | 3.35 | 2.22 |
| formulation | | | |

Statistical analysis of the data shows that the microparticle formulation group has improvement in the overall grades of the femoral condyles. The mean total score for the microparticle formulation group is lower than untreated or treatment with injection vehicle alone. Also, the standard error of the means is lower. Overall, the femoral condyle total scores are lower and tighter indicating a disease-modifying treatment of osteoarthritis. Therefore, microparticles in an injection vehicle, without added therapeutic agents, may be used as an intra-articularly delivered disease modifying treatment for osteoarthritis (OA).

### Example 4

### Treatment of Human Patient with Composition and Method of the Present Invention

A patient diagnosed with osteoarthritis in a knee is prepared for injection of a therapeutically effective dose of the composition of the present invention as described in Example 2. The patient's knee is palpated by the administering health care professional to determine an optimal site to insert a hypodermic needle into the joint in order to access the intra-articular space. The patient's knee is swabbed with a conventional disinfecting agent in a conventional manner. The composition is loaded into a conventional sterile syringe having a conventional sterile hypodermic needle. The needle is inserted through the skin of the pre-selected site into the joint until the intra-articular space is accessed. The health care professional then injects the composition through the needle in a conventional manner such that the contents of the syringe are completely injected into the intra-articular space. The health care professional then withdraws the needle, and treats the entry wound in a conventional manner, thus completing the procedure. The patient subsequently experiences relief of symptoms associated with the OA knee.

The novel method and compositions of the present invention for treating osteoarthritis have many advantages. The advantages include avoiding disadvantageous systemic side effects associated with oral delivery of therapeutic agents, therapeutic agents are not needed to achieve a therapeutic affect and treatment is sustained as a result of the slow degradation of microparticles.

Although this invention has been shown and described with respect to detailed embodiments thereof, it will be understood by those skilled in the art that various changes in form and detail thereof may be made without departing from the spirit and scope of the claimed invention.

We claim:

1. A method of treatment of osteoarthritis, comprising providing a sterile composition, said composition comprising biodegradable microparticles, wherein said biodegradable microparticles further comprise a polymer selected from the group consisting of poly(lactic acid), poly(glycolic acid), and copolymers of lactic acid and glycolic acid; and a carrier vehicle wherein said carrier vehicle further comprises an aqueous solution and a viscosity enhancer, wherein the viscosity enhancer is selected from the group consisting of hyaluronic acid and sodium hyaluronate; and,
delivering said composition into the intra-articular space of a joint.

2. The method of claim 1, wherein the joint is selected from the group consisting of hips, knees, shoulders, ankles, elbows, wrists, toes, fingers, and spine.

3. The method of claim 1, wherein the intra-articular space is the synovial fluid containing portion of the joint.

4-6. (canceled)

7. The method of claim 1 wherein the polymers are endcapped with a hydrophobic group.

8. The method of claim 7, wherein the hydrophobic group is lauryl ester or methoxy.

9. The method of claim 1, wherein the composition is delivered by injection through a hypodermic needle.

10. The method of claim 9, wherein the hypodermic needle has a size of about 16 gauge to about 24 gauge.

11. The method of claim 1, wherein the mean particle size is about 5 microns to about 150 microns.

12. The method of claim 1, wherein the mean particle size is about 10 microns to about 100 microns.

13. The method of claim 1, wherein the mean particle size is about 35 microns to 45 microns.

14-16. (canceled)

17. The method of claim 14, wherein the concentration of viscosity enhancer in the composition is about 0.5 wt. percent to about 5 wt. percent.

18. The method of claim 1, wherein the concentration of viscosity enhancer in the composition solution is about 1 wt. percent to about 3 wt. percent.

19. The method of claim 1, wherein the concentration of viscosity enhancer in the composition is about 1.5 wt. percent to about 2.5 wt. percent.

20. The method of claim 1, wherein the composition comprises about 1 wt. percent to about 20 wt. percent of biodegradable microparticles.

21. The method of claim 1, wherein the composition comprises about 1.5 wt. percent to about 9 wt. percent of biodegradable microparticles.

22-27. (canceled)
